(19) United States
(12) Patent Application Publication
(10) Pub. No.: US 2007/0254280 A1
(43) Pub. Date: Nov. 1, 2007

(54) Method of Identifying Characteristic of Molecules

(75) Inventors: Preben Lexow, Oslo (NO); Erland Ragnhildstveit, Oslo (NO)

Correspondence Address: MARSHALL, GERSTEIN & BORUN LLP, 233 S. WACKER DRIVE, SUITE 6300, SEARS TOWER, CHICAGO, IL, 60606, US

(73) Assignee: LINGVITAE AS, OSLO (NO)

(21) Appl. No.: 10/553,505

(22) PCT Filed: Apr. 16, 2004
(86) PCT No.: PCT/GB04/01665

§ 371 Date: Jan. 11, 2006

(30) Foreign Application Priority Data
Apr. 16, 2003 (GB) 0308852.3

(51) Int. Cl. C12Q 1/68

(52) U.S. Cl. 435/6

(57) ABSTRACT

The present invention is a method for identifying one or more characteristics of a target molecule, and comprises the steps of: converting the characteristics of the molecule into a polynucleotide of defined sequence, wherein each characteristic is represented by at least one distinct unit on the polynucleotide, the unit comprising at least a single base; contacting the polynucleotide with at least one of the nucleotides dATP, dTTP (dUTP), dGTP and dCTP, under conditions that permit the polymerisation reaction to proceed, wherein the at least one nucleotide comprises a detectable label specific for the nucleotide; removing any non-incorporated nucleotides and detecting any incorporation events; removing any labels; and repeating steps (ii) to (iv) to thereby identify the different units, and thereby the characteristics of the molecule.

### FIELD OF THE INVENTION

This invention relates to methods for identifying the characteristics of molecules. In particular, the invention relates to methods for determining the sequence of a polynucleotide.

### BACKGROUND TO THE INVENTION

Advances in the study of molecules have been led, in part, by improvement in technologies used to characterise the molecules or their biological reactions. In particular, the study of the nucleic acids DNA and RNA has benefited from developing technologies used for sequence analysis and the study of hybridisation events.

The principal method in general use for large-scale DNA sequencing is the chain termination method. This method was first developed by Sanger and Coulson (Sanger et al., Proc. Natl. Acad. Sci. USA, 1977; 74: 5463-5467), and relies on the use of dideoxy derivatives of the four nucleotides which are incorporated into the nascent polynucleotide chain in a polymerase reaction. Upon incorporation, the dideoxy derivatives terminate the polymerase reaction and the products are then separated by gel electrophoresis and analysed to reveal the position at which the particular dideoxy derivative was incorporated into the chain.

Although this method is widely used and produces reliable results, it is recognised that it is slow, labour-intensive and expensive.

U.S. Pat. No. 5,302,509 discloses a method to sequence a polynucleotide immobilised on a solid support. The method relies on the incorporation of 3-blocked bases A, G, C and T having a different fluorescent label to the immobilised polynucleotide, in the presence of DNA polymerase. The polymerase incorporates a base complementary to the target polynucleotide, but is prevented from further addition by the 3′-blocking group. The label of the incorporated base can then be determined and the blocking group removed by chemical cleavage to allow further polymerisation to occur. However, the need to remove the blocking groups in this manner is time-consuming and must be performed with high efficiency.

WO-A-00/39333 describes a method for sequencing polynucleotide by converting the sequence of a target polynucleotide into a second polynucleotide having a defined sequence and positional information contained-therein. The sequence information of the target is said to be “magnified” in the second polynucleotide, allowing greater ease of distinguishing between the individual bases on the target molecule. This is achieved using “magnifying tags” which are predetermined nucleic acid sequences. Each of the bases adenine, cytosine, guanine and thymine on the target molecule is represented by an individual magnifying tag, converting the original target sequence into a magnified sequence. Conventional techniques may then be used to determine the order of the magnifying tags, and thereby determining the specific sequence on the target polynucleotide.

In a preferred sequencing method, each magnifying tag comprises a label, e.g. a fluorescent label, which may then be identified and used to characterise the magnifying tag.

Although the method disclosed in this patent publication has many advantages, there is still a need for improved methods for sequencing target polynucleotides.

### SUMMARY OF THE INVENTION

The present invention is based on the realisation that individual characteristics of molecules can be converted into a defined polynucleotide sequence and that this defined sequence can be characterised by the incorporation of detectable labels.

According to a first aspect of the invention, a method for identifying a series of characteristics of a molecule comprises the steps of:

(i) converting the characteristics of the molecule into a polynucleotide of defined sequence, wherein each characteristic is represented by at least one distinct unit on the polynucleotide, the unit comprising at least a single base;

(ii) contacting the polynucleotide with at least one of the nucleotides dATP, dTTP (dUTP), dGTP and dCTP, under conditions that permit the polymerisation reaction to proceed, wherein the at least one nucleotide comprises a detectable label specific for the nucleotide;

(iii) removing any non-incorporated nucleotides and detecting any incorporation events;

(iv) removing any labels; and

(v) repeating steps (ii) to (iv) to thereby identify the different units, and thereby the characteristics of the molecule.

The invention permits the rapid identification of each distinct unit on the polynucleotide, which in turn allows each distinct characteristic on the target molecule to be characterised.

The method is particularly suitable for identifying one or more bases present on a target polynucleotide (molecule), for example in determining the sequence of the target polynucleotide.

In a second aspect of the invention, a method for identifying a series of characteristics of a molecule comprises the steps of:

(i) converting the characteristics of the molecule into a polynucleotide of defined sequence, wherein each characteristic is represented by at least one distinct unit comprising at least a two base sequence;

(ii) contacting the polynucleotide with an oligonucleotide under hybridising conditions, the oligonucleotide being complementary to a unit on the polynucleotide and being detectably labelled;

(iii) removing any non-hybridised oligonucleotides and detecting an hybridisation event;

(iv) removing any label(s); and

(v) optionally repeating steps (ii) to (iv) to thereby identify the different units, and thereby the characteristics of the target polynucleotide.

### DESCRIPTION OF THE DRAWINGS

The present invention is described with reference to the accompanying drawings, wherein:

FIG. 1 is a schematic illustration of the “units” of sequence that represent individual bases on a target polynucleotide;

FIG. 2 is a schematic illustration of the apparatus used to detect fluorescent signals generated during the method;

FIG. 3 is a schematic illustration of the results obtained during the polymerase extension reaction; and

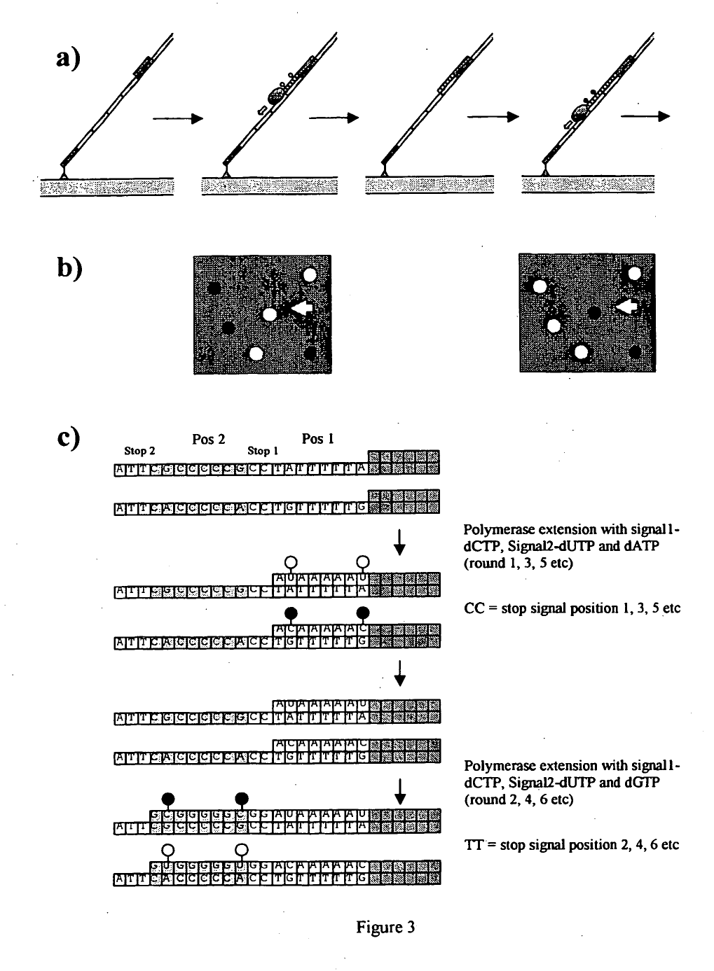

FIG. 4 is a schematic illustration of the method steps resulting in the conversion of the target polynucleotide into a defined second polynucleotide.

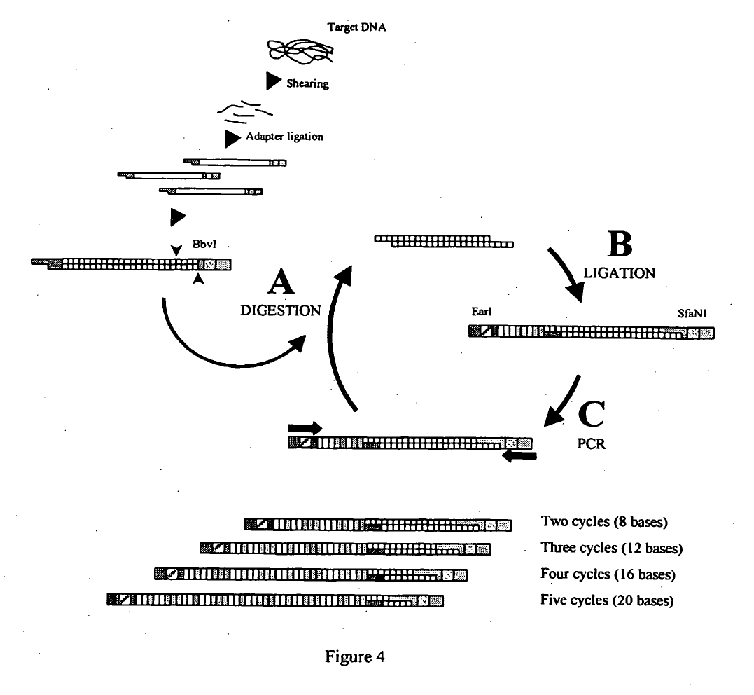

### DESCRIPTION OF THE INVENTION

The invention relies on the conversion of a target molecule into a polynucleotide having distinct, defined, units of nucleic acid sequence, each unit, or unique combination of units, being representative of a particular characteristic on the target molecule.

Each unit (and hence each characteristic) can be determined by making use of the polymerase reaction to incorporate detectably-labelled complementary nucleotides onto each unit. Detecting the label for each incorporation event characterises the unit. In a preferred embodiment detailed below, the invention utilises a specific design for each unit, to permit incorporation to occur in a highly controlled manner, allowing a highly automated analysis to take place.

The term “molecule” refers to any biological or chemical molecule. Preferred molecules are biological molecules, including polynucleotides, eg DNA.

The term “polynucleotide” is well known in the art and is used to refer to a series of linked nucleic acid molecules, e.g. DNA or RNA. Nucleic acid mimics, e.g. PNA, LNA (locked nucleicacid) and 2′-O-methRNA are also within the scope of the invention.

The reference to the bases A, T(U), G and C, relates to the nucleotide bases adenine, thymine (uracil), guanine and cytosine, as will be appreciated in the art. Uracil replaces thymine when the polynucleotide is RNA, or it can be introduced into DNA using dUTP, again as well understood in the art. Similarly, reference to the nucleotides “dATP”, “dTTP”, “dUTP”, “dGTP” and “dCTP”, relates to the corresponding deoxynucleotide triphosphates, as will be evident to the skilled person.

It will be appreciated by the skilled person that base or nucleotide analogues are known and are within the scope of the present invention. The analogues retain the ability to bind (hybridise) specifically to their complement.

The polynucleotide is said to comprise distinct “units” of nucleic acid sequence. Each characteristic on the target is represented by a distinct and predefined unit, or unique combination of units. For example, if the target molecule is a polynucleotide, each base on the target polynucleotide is represented by a distinct and predefined unit. Each unit will preferably comprise two or more nucleotide bases, preferably from 2 to 50 bases, more preferably 2 to 20 bases and most preferably 4 to 10 bases, e.g. 6 bases. There are preferably at least two different bases contained in each unit. In a preferred embodiment there are three different bases in each unit. The design of the units is such that it will be possible to distinguish the different units during a “read-out” step, involving either the incorporation of detectably labelled nucleotides in a polymerisation reaction, or on hybridisation of complementary oligonucleotides. For example, each characteristic on the target is represented by a series of bases in a unit, where one base will be complementary to a labelled nucleotide introduced during the read-out step, one base will act as a “spacer” to provide separation between incorporated labels, and one base will act as a stop signal.

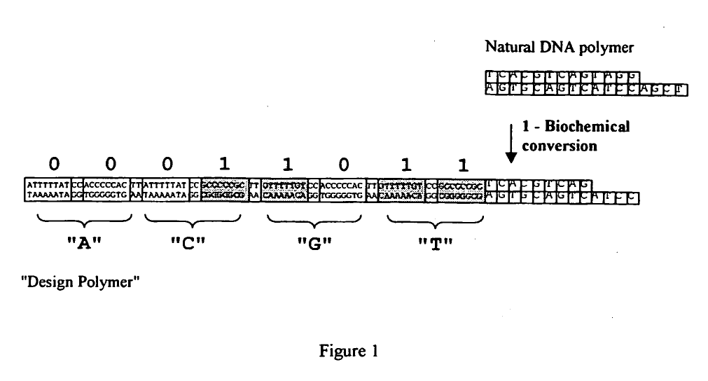

In a preferred embodiment, when the target molecule is a polynucleotide, two units of distinct sequence are used to represent all of the four possible bases on the target polynucleotide. According to this embodiment, the two units can be used as a binary system, with one unit representing “0” and the other representing “1”. Each base on the target is characterised by a combination of the two units. For example, adenine may be represented by “0”+“0”, cytosine by “0”+“1”, guanine by “1”+“0” and thymine by “1”+“1”, as shown in FIG. 1. It is necessary to distinguish between the units, and so a “stop” signal can be incorporated into each unit. It is also preferable to use different units representing “1” and “0”, depending on whether the base on the target (template) polynucleotide is in an odd or even numbered position.

This is demonstrated as follows:

Odd numbered template sequence:

| | |
|---|---|
| “0” | TTTTTTA(CCC) |
| “1” | TTTTTTG(CCC) |

Even numbered template sequence:

| | |
|---|---|
| “0” | CCCCCCA(TTT) |
| “1” | CCCCCCG(TTT) |

In this example, the underlined base is the target for labelled nucleotides in a polymerase reaction, the bases in parentheses are used as a stop signal, and the remaining bases are to provide separation between the labels.

In odd numbered positions (1, 3, 5, etc) the nucleotide mix, introduced during the polymerase reaction, consists of Fluor X-dUTP, Fluor Y-dCTP and dATP (dGTP is missing from the mix). The complementary base for Fluor Y is missing for “0”, and the complementary base for Fluor X is missing for “1”. Accordingly, during a polymerase reaction, if the unit “0” is present, it will be possible to detect this by monitoring for Fluor X, and if “1” is present, by monitoring for Fluor Y.

In all even numbered positions (2, 4, 6, etc) the nucleotide mix consists of the same two fluor-labelled nucleotides, but dGTP is used, not dATP, and one or more T bases define the stop signal.

After each unit has been “read” it is possible to restart the process by introducing the missing complementary nucleotide (eg. either dGTP or dATP) to allow incorporation at the stop sequence. Non-incorporated nucleotides are washed away prior to the next read-out step.

The method of the invention may be used to determine the sequence of a target polynucleotide, or may be used to identify the presence and/or type of nucleotide present at a specific position on the target polynucleotide. For example, the method may be used to identify whether specific single nucleotide polymorphisms are present on a target polynucleotide. The method may also be used for restriction mapping and haplotyping.

The different characteristics of many molecules can be dertermined using the present invention. In addition to sequencing procedures the present method may be used to identify binding characteristics of molecules, eg., protein binding properties, enzymatic properties or other chemical or biochemical property. The different characteristics may be identified by carrying out reactions to test for each characteristic and associating a specific polynucleotide unit to each molecule that undergoes reaction. For example, if the protein-binding characteristic of a molecule is to be studied, a reaction can be performed so that the molecule and a suitable protein are brought into contact under appropriate conditions and those molecules that bind to the protein are retained in a reaction compartment, and non-protein-binding molecules are removed. A specific polynucleotide unit may then be incorporated onto the molecule, to characterise the specific protein-binding property. Further binding studies using different proteins of interest may then be carried out, and subsequent binding events characterised by the sequential incorporation of polynucleotide units, to thereby form a single polynucleotide of multiple defined units.

The attachment of a polynucleotide unit onto a molecule may be carried out by various means, depending on the nature of the molecule. If the molecule is not a polynucleotide, then attachment to the molecule may be via a first linker molecule that binds to the molecule and the first polynucleotide unit. It is preferable if the attachment is via a covalent bond and so a chemical linkage is preferred. Suitable methods for binding a polynucleotide to a non-polynucleotide are known in the art.

Attachment of subsequent units can utilise base-base complementarity, so that a subsequent unit hybridises within a portion of the preceding unit and is ligated in a ligation reaction. This is described in WO-A-00/39333.

The target molecule may be converted into the defined units using methods known in the art. For example, the conversion method disclosed in WO-A-00/39333 (the content of which is incorporated herein by reference), using restriction enzymes, may be adopted. For example, if the target is a polynucleotide, the target polynucleotide may be ligated into a vector which carries a class IIS restriction site close to the point of insertion, or the target polynucleotide may be engineered to contain such a site. The appropriate class IIS restriction enzyme is then used to cleave the restriction site, resulting in an overhang in the target sequence.

Appropriate adapters which contain one or more of the units may then be used to bind to one or more of the bases of the overhang. Once the overhang of the adapter and the cleaved vector have, been hybridised, these molecules may be ligated. This will only be achieved where full complementarity along the full extent of the overhang is achieved. Blunt-end ligation may then be effected to join the other end of the adapter to the vector. By appropriate placement of a further class II restriction site (or other appropriate restriction enzyme site), which may be same or different to the previously used enzyme, cleavage may be effected such that an overhang is created in the target sequence downstream of the sequence to which the first adapter was directed. In this way, adjacent or overlapping sequences may be consecutively converted into sequences carrying the units of defined sequence.

Using this conversion system, the defined units are formed using the binary system, wherein two consecutive units are used to define a particular base on the target polynucleotide.

Having converted the target sequence into the sequential units of the second polynucleotide, the sequence of the units may then be determined, to thereby determine the target polynucleotide sequence.

This may be achieved as discussed above using the polymerase reaction to incorporate bases complementary to those on the second polynucleotide, using either selected, detectably-labelled nucleotides or nucleotides that incorporate a group for subsequent indirect labelling, and monitoring any incorporation event.

The polymerase reaction is preferably carried out under conditions that permit the controlled incorporation of complementary nucleotides one unit at a time. This enables each unit to be categorised by the detection of an incorporated label. As each unit preferably comprises a “stop” sequence, it is possible to control incorporation by supplying only those nucleotides required for incorporation onto the first unit, as described above. As each unit is recognised by a specific label, it is possible to distinguish between two different units (0 and 1) within each cycle. This enables detection of any incorporated label, and allows the identification and position of the unit to be determined.

The method may be carried out as follows:

(i) contacting the polynucleotide comprising the defined units with at least one of the nucleotides dATP, dTTP, dGTP and dCTP, under conditions that permit the polymerisation reaction to proceed, wherein the at least one nucleotide comprises a detectable label specific for that nucleotide;

(ii) removing any non-incorporated nucleotides and detecting any incorporation events;

(iii) removing the labels from incorporated nucleotide; and

(iv) repeating steps ii) to iv), to thereby identify the different units, and thereby the sequence of the target polynucleotide.

The number of different nucleotides required in step (i) of each cycle will be dependent on the design of the units. If each unit comprises only one base type, then only one nucleotide (detectably labelled) is required. However, if two bases are utilised (one as a target for the detectably labelled nucleotide and one to provide a gap between different target bases) then two nucleotides will be required (one to bind to the target base and one to “fill in” the bases between the target bases).

The use of a base as a stop signal allows the detection steps to be performed without the requirement for blocked nucleotides to prevent uncontrolled incorporation during the polymerase reaction. The stop signal is effective as the complement for the “stop” base is absent from the polymerase mix. Therefore, each unit can be characterised before a “fill-in” step is performed, using the missing nucleotide, to incorporate a complement to the stop base, which allows the next unit to be charcterised. This is carried out after the detection step. The “stop” base of one unit will not be of the same type as the first base of the subsequent unit. This ensures that the “fill-in” procedure does not progress to the next unit, Non-incorporated nucleotides used in the “fill-in” procedure can then be removed, and the next unit can then be characterised.

The choice of polymerase and detectable label will be apparent to the skilled person. The following is used as a guide only:
a) Klenow and Klenow (exo-) can efficiently incorporate Tetramethylrhodamine-4-dUTP and Rhodamin-110-dCTP (Amersham Pharmacia Biotech) (Brakmann and Nieckchen, 2001, Brakmann and Lobermann, 2000).
b) Vent, Taq and Tgo DNA polymerase can efficiently incorporate dioxigenin and fluorophores like AMCA, Tetramethylrhodamin, fluorescein and Cy5 without spacing at least up to a few positions (Augustin et al, (provide reference?) 2001).
c) T4 DNA polymerase is efficient in filling-in fluorophore labelled nucleotides.

The preferred polymerases are Klenow Large fragment (exo-) and T4 DNA polymerase.

In a preferred embodiment, after the conversion step, multiple polynucleotides (comprising the defined units) are immobilised on a support material. This places each polynucleotide in a fixed position, and allows the sequence of each polynucleotide to be determined by aligning consecutive images of the support material to establish the order in which the labels were detected.

Polynucleotides may be attached to support materials by recognised means, including the use of biotin-avidin interactions. Methods for immobilising polynucleotides on support materials are well known in the art, and include photolithographic techniques and techniques that rely on “spotting” individual polynucleotides in defined positions on a support material. Immobilisation may also be carried out by the random distribution of polynucleotides on microbeads, nanoparticles and planar surfaces.

Immobilisation may be by specific covalent or non-covalent interactions. The interaction should be sufficient to maintain the polynucleotides on the support during washing steps to remove unwanted reaction components. Immobilisation will preferably be at either the 5′ or 3′ position, so that the polynucleotide is attached to the support at the end only. However, the polynucleotide may be attached to the support at any position along its length, the attachment acting to tether the polynucleotide to the support.

The skilled person will appreciate the appropriate means to immobilise the polynucleotide to the support material. Suitable coatings may be applied to the support to facilitate immobilisation, as will be appreciated by the skilled person. Suitable coatings include epoxy coatings (eg. 3-glycidyloxypropyltrimethoxysilane), superaldehyde coating, mercaptosilane, and isothiocyanate. Alternatively, several linker groups may be used, including PAMAM dendritic structures (Benters et al., Chem Biochem., 2001; 2: 686-694) and the immobilisation linkers described in Zhao et al., Nucleic Acids Research, 2001; 29(4): 955-959.

Suitable support materials are known in the art, and include glass slides, ceramic and silicon surfaces and plastics materials. The support is usually a flat (planar) surface.

The second polynucleotide may be immobilised on the support material to form polynucleotide arrays which may form a random or ordered pattern on the solid support. Preferably, the arrays that are used are single molecule arrays that comprise polynucieotides in distinct optically resolvable areas, e.g. as disclosed in WO-A-00/06770, the contents which incorporated herein by reference.

To carry out the polymerase reaction it will usually be necessary to first anneal a primer sequence to the polynucleotide, the primer sequence being recognised by the polymerase enzyme and acting as an initiation site for the subsequent extension of the complementary strand. The primer sequence may be added as a separate component with respect to the polynucleotide, which comprises a complementary sequence that allows the primer to anneal.

Other conditions necessary for carrying out the polymerase reaction, including temperature, pH, buffer compositions etc., will be apparent to those skilled in the art. The polymerisation step is likely to proceed for a time sufficient to allow incorporation of bases to the first unit. Non-incorporated nucleotides are then removed, for example, by subjecting the array to a washing step, and detection of the incorporated labels may then be carried out.

An alternative strategy is to use short detectably labelled oligonucleotides to hybridise to the units on the polynucleotide, and to detect any hybridisation event.

The short oligonucleotides have a sequence complementary to specific units of the polynucleotide. For example, if a binary system is used and each characteristic is defined by a different combination of units (one representing “0” and one representing “1”) the invention will require an oligonucleotide specific for the “1” unit. In this embodiment, selective hybridisation of oligonucleotides can be achieved by designing each unit to be of a different polynucleotide sequence with respect to other units. This ensures that a hybridisation event will only occur if the specific unit is present, and the detection of hybridisation events identifies the characteristics on the target molecule.

In a preferred embodiment, the label is a fluorescent moiety. Many examples of fluorophores that may be used are known in the prior art, and include:
Alexa dyes (Molecular Probes)
BODIPY dyes (Molecular Probes)
Cyanine dyes (Amersham Biosciences Ltd.)
Tetramethylrhodamine (Perkin Elmer, Molecular Probes, Roche Diagnostics)
Coumarin (Perkin Elmer)
Texas Red (Molecular Probes)
Fluorescein (Perkin Elmer, Molecular Probes, Roche Diagnostics)

The attachment of a suitable fluorophore to a nucleotide can be carried out by conventional means. Suitably labelled nucleotides are also available from commercial sources. The label is attached in a way that permits removal, after the detection step. This may be carried out by any conventional method, including:
I. Attacking the signal itself:
a) Bleaching

i) Photobleaching

ii) Chemical bleaching
b) Quenching of fluorescence

i) By antibodies raised against the fluor (eg. anti-fluorescein, anti-Oregon green)

ii) By FRET (the incorporation of a quencher next to a signal can be used to quench the signal, eg. Taqman strategy)
c) Cleavage of signal

i) Chemical cleavage (eg. reduction of a disulfide bridge between the base and the signal)

ii) Photocleavage (eg. introduction of a nitrobenzyl or tert-butylketon group)

iii) Enzymatic (eg. α-chymotryspin digestion of peptide linker)
II. The signal bearing nucleotide:
a) Exonucleolytic removal

i) 3′-5′ Exonucleolytic degradation of filled-in nucleotides (eg. exonuclease III or by activating the 3′-5′ exonucleolytic activity of DNA polymerase when there is an absence of certain nucleotides)
b) Restriction enzyme digestion

i) Digestion of double-stranded DNA bearing the signal (eg. Apal, Dral, Smal sites which can be incorporated at the stop signals).

An alternative to the use of labels that permit removal, is to use inactivated labels that are reactivated during a biochemical process.

The preferred method is by photo or chemical cleavage.

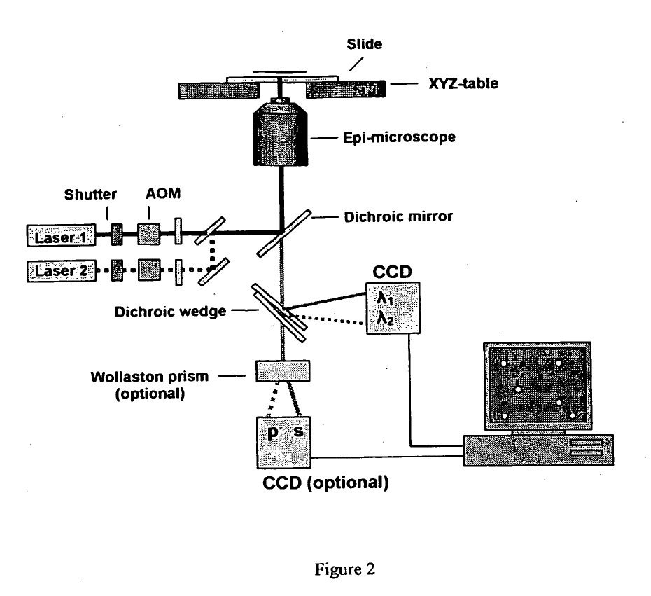

When the label is a fluorophore, the fluorescent signal generated on incorporation may be measured by optical means, e.g. by a confocal microscope. Alternatively, a sensitive 2-D detector, such as a charge-coupled detector (CCD), can be used to visualise the individual signals generated, as shown in FIG. 2.

The general set-up for optical detection is as follows:

| | |
|---|---|
| Microscope: | Epi-fluorescence |
| Objective: | Oil emersion (100×, 1.3 NA) |
| Light source: | Lasers or lamp |
| Filters: | Bandpass |
| Mirrors: | Dichroic mirror and dichroic wedge |
| Detectors: | Photomultiplier tubes (PMT) or CCD camera |

Variants may also be used, including:

A. Total Internal Reflection Fluorescence Microscopy (TIRFM)

| | |
|---|---|
| Light source: | One or more lasers |
| Background control: | No pinhole required |

Detection: CCD camera (video and digital imaging systems)

B. Confocal Laser Scanning Microscopy (CLSM)

| | |
|---|---|
| Light source: | One or more lasers |
| Background | One or several pinhole apertures |
| reduction: | |
| Detection: | a) A single pinhole: Photomultiplier tube (PMT) |
| | detectors for different fluorescent wavelengths [The |
| | final image is built up point by point and over time by |
| | a computer]. |
| | b) Several thousands pinholes (spinning Nipkow |
| | disk): CCD camera detection of image [The final |
| | image can be directly recorded by the camera] |

C. Two-Photon (TPLSM) and Multiphoton Laser Scanning Microscopy

| | |
|---|---|
| Light source: | One or more lasers |
| Background control: | No pinhole required |
| Detection: | CCD camera (video and digital imaging systems) |

The preferred methods are TIRFM and confocal microscopy.

The following Examples illustrate the invention.

### EXAMPLE 1

Primer extension:

A target polynucleotide is converted into a series of second polynucleotides using the methods disclosed in WO-A-00/39333. Four defined second polynucleotides are used to represent 0 and 1 units in both even and odd numbered positions. The 0- and 1-units have the sequence TTTTTTACCC and TTTTTTGCCC, respectively, in odd numbered positions, while their codings are CCCCCCATTT and CCCCCCGTTT, respectively, in even numbered positions.

5′-amino labeled single-stranded second polynucleotides are generated from double-stranded template (end product of the conversion) by asymmetric PCR using 5′-amino labeled primer, DNA polymerase and dNTPs. A common primer is annealed to the amino-labeled second polynucleotides and the molecule is immobilized to an expoxy-coated glass slide via the amino-group. Conditions are chosen to avoid aggregation of the molecules (e.g. low salt) and to ensure single molecule resolution by fluorescence microscopy.

A buffer solution “odd” containing Alexa-488-dUTP (or Cy3-dUTP), Alexa-647-dCTP (or Cy5-dCTP), dATP (dGTP missing) and DNA polymerase (Klenow or T4 DNA polymerase) is added to the slides. The fluorophore labeled nucleotides contain a photocleavable linker inserted between the fluorochrome and the base. The slides are incubated for a few minutes for the polymerase reaction to occur. After a washing procedure to remove DNA polymerase and unincorporated nucleotides, a series of images covering the entire slide are captured using TIR fluorescence microscopy and ICCD-camera detection. The label is removed by photocleavage (340 nm for 2-nitrobenzyl linker), and the slide is ready for a second round after a brief wash to remove the cleaved label. A buffer solution “even” containing exactly the same constituents as used in “odd” only with dGTP replacing dATP, is added to the slide to start the fill-in of position two. Detection and removal of signal proceeds as described for cycle one. By cycling between these two buffer systems, the units are determined in a controlled manner.

### EXAMPLE 2

Oligonucleotide Hybridisation:

The same target polynucleotide is sequenced using a method based on hybridisation. 0- and 1-units are built up from 15-20 bp sequences that define both the base on the target polynucleotide and its position. Thus, a second polynucleotide containing 40 units (i.e. 20 bp from the target polynucleotide) is built up from a repertoir of 2×40 different 15-20 bp sequences having similar melting characteristics.

5′-amino labeled single-stranded second polynucleotides are generated from double-stranded template (end product of the conversion) by asymmetric PCR using 5′-amino labeled primer, DNA polymerase and dNTPs. The second polynucleotides are immobilized to a glass slide via the amino-group, using a glass coating that can withstand several cycles of hybridization and denaturation (PAMAM dendrimer coated glass slide). Conditions are chosen to avoid aggregation of the molecules (e.g. low salt) and to ensure single molecule resolution by fluorescence microscopy.

Two different fluorophore-labeled oligonucleotides representing 0 and 1, respectively in position one are hybridised to the immobilised polynucleotides using stringent conditions (to avoid mis-hybridisation). After several stringent washes to remove unhybridised oligos, images are captured as described in Example 1.

1. A method for identifying a series of characteristics of a molecule comprising the steps of:
(i) converting the characteristics of the molecule into a polynucleotide of defined sequence, wherein each characteristic is represented by at least one distinct unit on the polynucleotide, the unit comprising at least a single base;
(ii) contacting the polynucleotide with at least one of the nucleotides dATP, dTTP (dUTP), dGTP and dCTP, under conditions that permit the polymerisation reaction to proceed, wherein the at least one nucleotide comprises a detectable label specific for the nucleotide;
(iii) removing any non-incorporated nucleotides and detecting any incorporation events;
(iv) removing any labels; and
(v) repeating steps (ii) to (iv) to thereby identify the different units, and thereby the characteristics of the molecule.

2. A method according to claim 1, wherein each unit on the polynucleotide comprises two or three of the different bases A, T(U), G and C, one of which represents a target for the subsequent incorporation of the detectably labelled nucleotide, and one represents a stop signal,
and wherein step (ii) is carried out in the presence of nucleotides complementary to the bases of the unit but in the absence of a nucleotide complementary to that of the stop signal.

3. A method according to claim 1 or claim 2, wherein consecutive units on the polynucleotide have a different base type as the target for the incorporation of a labelled nucleotide.

4. A method according to any preceding claim, wherein each unit comprises two bases of the same type as targets for the incorporation of labelled nucleotides, with the two bases optionally separated by one or more of a third base of a different type.

5. A method according to any preceding claim, wherein the molecule is a target polynucleotide.

6. A method according to claim 5, wherein the characteristic to be identified is the partial or complete sequence of the target polynucleotide.

7. A method according to any preceding claim, wherein the label is a fluorophore.

8. A method according to claim 7, wherein the fluorophore is Alexa-red or Alexa-green.

9. A method according to any preceding claim, wherein the polynucleotide of step (i) is immobilised on a support material.

10. A method according to claim 9, wherein the immobilised polynucleotide forms an array on the support material, the array having a density that permits individual resolution of a detectable label.

11. A method according to any preceding claim, wherein detection is carried out by optical microscopy.

12. A method according to claim 5 and any claim pendent to claim 5, wherein each of the bases A, T(U), G and C on the target polynucleotide is represented by a combination of two sequential units, with each base represented by a different combination of the two units.

13. A method for identifying one or more characteristics of a molecule comprising the steps of:
i) converting the characteristics of the molecule into a polynucleotide of defined sequence, wherein each characteristic is represented by at least one distinct unit comprising at least a two base sequence;
ii) contacting the polynucleotide with an oligonucleotide under hybridising conditions, the oligonucleotide being complementary to a unit on the polynucleotide and being detectably labelled;
iii) removing any non-hybridised oligonucleotides and detecting an hybridisation event;
iv) removing any label(s); and
v) optionally repeating steps (ii) to (iv) to thereby identify the different units, and thereby the characteristics of the target polynucleotide.

14. A method according to claim 13, wherein the molecule is a polynucleotide and the sequence of the target polynucleotide is determined.

15. A method according to claim 13 or claim 14, wherein the polynucleotide comprising the units is immobilised on a support material.

16. A method according to claim 15, wherein the immobilised polynucleotide forms an array on the support material, the array having a density that permits individual resolution of a detectable label on each polynucleotide.


SEQUENCE LISTING (11 sequences)

SEQ ID NO: 1 - DNA, 10 bp, Artificial sequence
  Synthetic peptide
ttttttaccc

SEQ ID NO: 2 - DNA, 10 bp, Artificial sequence
  Synthetic peptide
ttttttgccc

SEQ ID NO: 3 - DNA, 10 bp, Artificial sequence
  Synthetic peptide
ccccccattt

SEQ ID NO: 4 - DNA, 10 bp, Artificial sequence
  Synthetic peptide
ccccccgttt

SEQ ID NO: 5 - DNA, 87 bp, Artificial sequence
  Synthetic polymer
atttttatccacccccacttatttttatccgcccccgcttgtttttgtccacccccacttgtttttgtccgcccccgctcacgtcag

SEQ ID NO: 6 - DNA, 91 bp, Artificial sequence
  Synthetic polymer
taaaaataggtgggggtgaataaaaataggcgggggcgaacaaaaacaggtgggggtgaacaaaaacaggcgggggcgagtgcagtcatcc

SEQ ID NO: 7 - DNA, 21 bp, Artificial sequence
  Synthetic polymer
attcgcccccgcctattttta

SEQ ID NO: 8 - DNA, 21 bp, Artificial sequencre
  Synthetic polymer
attcacccccacctgtttttg

SEQ ID NO: 9 - DNA, 29 bp, Artificial sequence
  Synthetic polymer
  misc_feature (2)..(2): n=uracil
  misc_feature (8)..(8): n=uracil
anaaaaanattcgcccccgcctattttta

SEQ ID NO: 10 - DNA, 29 bp, Artificial sequence
  Synthetic polymer
  misc_feature (2)..(2): n=uracil
  misc_feature (8)..(8): n=uracil
anaaaaanattcgcccccgcctattttta

SEQ ID NO: 11 - DNA, 39 bp, Artificial sequence
  Synthetic polymer
  misc_feature (12)..(12): n=uracil
  misc_feature (18)..(18): n=uracil
gcgggggcgganaaaaanattcgcccccgcctattttta